(19) United States
(12) Patent Application Publication
(10) Pub. No.: US 2010/0255447 A1
(43) Pub. Date: Oct. 7, 2010

(54) ADVANCED BIO-COMPATIBLE POLYMER SURFACE COATINGS FOR IMPLANTS AND TISSUE ENGINEERING SCAFFOLDS

(75) Inventors: ALEXANDRU S. BIRIS, Little Rock, AR (US); PEDER JENSEN, Little Rock, AR (US); GANESH KANNARPADY, Little Rock, AR (US)

Correspondence Address: FOLEY AND LARDNER LLP;SUITE 500, 3000 K STREET NW, WASHINGTON, DC, 20007, US

(73) Assignee: University of Arkansas

(21) Appl. No.: 12/604,964

(22) Filed: Oct. 23, 2009

Related U.S. Application Data
(60) Provisional application No. 61/212,110, filed on Apr. 7, 2009.

(51) Int. Cl. A61C 8/00; A61B 19/02; A61F 2/02; A61F 2/28; C04B 35/00; C09D 1/00; C09D 189/00; C03C 3/00; B32B 5/16; B05D 3/00; C23C 14/34; C23F 1/00; A61L 2/08; A01N 43/20; C12N 5/00

(52) U.S. Cl. 433/201.1; 206/438; 623/23.76; 623/23.6; 523/105; 501/1; 106/286.1; 106/286.4; 106/124.4; 106/286.8; 501/11; 428/323; 427/2.24; 427/2.26; 427/2.25; 204/192.15; 216/94; 422/22; 514/475; 435/402

(57) ABSTRACT

Disclosed herein are methodologies and compositions for coating materials, which can be used in a variety of biological applications.

### CROSS-REFERENCE TO RELATED APPLICATION

This application claims the benefit of U.S. Provisional Application No. 61/212,110, filed Apr. 7, 2009.

### INTRODUCTION

Annually, millions of implants are placed inside of organisms, including humans and animals. Most of these implants serve complex roles including but not limited to tissue replacement, mechanical support, tissue generation, cosmetic enhancement, complete or partial limb replacement, joint replacement, tooth replacement, spine reconstruction, defibrillators/pacemakers, in addition to electrodes and wires.

Because most implants are made of metals, metal oxides, polymeric materials or tissue components obtained from animals or humans, implant bio-compatibility poses a limitation in many applications as implants need to perform complex functions in the human body and their binding to the host tissue is crucial. For example, dental implants need to adhere very strongly to the jaw bone. It is also important for implant surfaces to prevent or reduce biofilm formation, which leads to infection and implant failure. Likewise, implants used for hip or knee replacements must integrate very closely and strongly with the bone structure of the skeleton. To meet these requirements, implants are constructed from bio-compatible materials such as titanium, polymeric materials, or ceramic materials. Still a relatively large number of such materials are being rejected every year by human patients and in most of these cases, the reasons are related to the poor integration of the implant surface with the bone/tissue structure and the growth and adherence of cells at the implant surface. Furthermore, many implants are lost due to infections caused by growth of biofilm on the implant surface.

### SUMMARY

Embodiments herein include but are not limited to methods, devices, compositions, kits, materials, tools, instruments, reagents, products, compounds, pharmaceuticals, arrays, computer-implemented algorithms, and computer-implemented methods.

In one aspect, there is provided a coating comprising micro and/or nano materials deposited on a surface. In one embodiment, the coating is deposited onto a device, such as a medical device. In a further embodiment, a medical device is implantable and can be delivered into a host organism, such as a human or animal, or used in vitro. The medical device may comprise plasmids, genes, nucleic acids, or a DNA or RNA virus. The micro and/or nano materials comprise material having a size from about 0.5 nm to about 50 mm.

In another embodiment, the coating covers at least a portion of said device. The coating comprises natural or synthetic polymer, metal, metal oxide, oxide, metal nitride, borate, ceramic, zirconia, allograft hard tissue, allograft soft tissue, xenograft hard tissue, xenograft soft tissue, carbon nanostructure, carbon, glasses, natural, or biocompatible material. The coating is capable of performing at least one of preventing oxidation; decreasing toxicity; treating infection, preventing infection; promoting cell adhesion; preventing biofilm formation, inhibiting biofilm formation; promoting cell proliferation; promoting binding with a biological or non-biological system, increasing or decreasing a cell function; delivering a drug and/or bioactive agent, or ensuring a better integration of a material into the host tissue.

In another embodiment, the coating is deposited by one or more of ion beam deposition, electron beam deposition, pulsed laser deposition, thermal sputtering and deposition, RF sputtering, laser etching, glancing angle deposition, electrospray, chemical vapor deposition, physical vapor deposition, or molecular epitaxy. The coating may be produced by self assembly and self formation during deposition.

In another embodiment, at least one surface of the coating may undergo plasma/ion treatment to induce the formation of surface charges that enhance the binding of bioactive agents, growth factors, and/or drugs, promote cell adhesion and proliferation, and/or increase the hydrophilic nature of the surface.

In another embodiment, the coating comprises nanoparticles and microparticles. In other embodiments, the coating comprises one or more layers of nanoparticles and/or microparticles. In still other embodiments, the one or more layers comprises a single type of nanoparticle and/or microparticle, or a combination of more than one type of nanoparticle and/or microparticle. Further, one or more layers comprises silver nanoparticles. In another embodiment, one or more layers comprises a combination of metal, nanoparticles, metal oxides, carbon nanotubes, polymeric nanoparticles, ceramics, calcium phosphate, collagen, and/or hydroxyapatite nanoparticles. In other embodiments, the coating is biodegradable and/or biocompatible, and nanoparticles can be released from said nanoparticle composition as each layer degrades. In other embodiments, a drug, growth factor, and/or bioactive agent is deposited within at least one layer and/or on the surface layer of said coating. In other embodiments, the nanoparticles comprise gold, silver, metals, oxides, carbon nanostructures (single, double, multi walled nanotubes, graphenes, fullerenes, nanofibers), hydroxyapatite, zirconia, natural or synthetic polymers, ceramics, or metal oxide.

In other embodiments, the medical device is an orthopedic implant, dental implant, veterinary prosthetic device, graft, needle, bone material, contact lens, catheter, ear tube, endotracheal tube, stent, shunt, scaffold, or tissue engineering matrix, breast implant, allograft hard tissue, allograft soft tissue, xenograft hard tissue, xenograft soft tissue, polymeric mesh, or ceramic mesh. The orthopedic implant is a hip implant, knee implant, shoulder implant, plate, pin, screw, wire, or rod. The dental implant is an abutment, healing screw, or cover screw. The veterinary prosthetic device is an implant, pin, screw, plate, or rod.

In other embodiments, the coating comprises one or more layers comprise at least one of a protein, amino acid, enzyme, nucleic acid, bioactive agent, growth factor, drug, antibiotic, nucleic acid, hormone, antibody, or agent that inhibits biofilm formation and may be released as layer(s) degrade. In a further embodiment, the growth factor is a bone morphogenic protein capable of promoting bone formation adjacent to or on the surface of a device. In another embodiment, the bioactive agent is in or on the surface coating of a medical device and affects adjacent tissue or cells in at least one or more of bone formation, protein synthesis, gene, expression, cell proliferation, mitosis, DNA transcription, hormone production, enzyme production, cell death, gene delivery, or drug delivery. In a still further embodiment, the bioactive agent may be linked to said nanoparticles and the linkage may be a covalent, ionic, hydrogen bond, sulfide bond, or polar covalent bond.

In another embodiment, a structured surface can be prepared by at least one of flame spraying, acid etching, grit blasting, casting-in, forging-in, laser texturing, micromachining, plasma treatment, ion bombardment, physical vapor deposition, or chemical vapor deposition

In another aspect, there is provided a method for inhibiting biofilm formation on a medical implant, comprising coating said implant with an agent(s) that prevents biofilm formation and/or growth of bacteria. In one embodiment, a biofilm is a bacterial, fungal, or protozoan biofilm. In another embodiment, a medical implant is an orthopedic or dental implant, graft, needle, bone material, contact lens, catheter, ear tube, endotracheal tube, stent, shunt, breast implant, scaffold, allograft hard tissue, allograft soft tissue, xenograft hard tissue, xenograft soft tissue ,or tissue engineering matrix. In another embodiment, the agent is triclosan, iodine, silver, phenol, chloride compounds, fluoride compounds, iodine, quaternary ammonium compounds, chlorhexidine, antibiotic, antifungal agent, or any other agent that inhibits biofilm formation and/or growth.

In another aspect, there is provided a method for inhibiting microbial colonization on a medical device or implant, comprising coating said device or implant with an agent or surface treatment that prevents microbial colonization. In one embodiment, the device or implant is a dental implant, healing screw or cover screw for a dental implant, orthopedic implant, veterinary implant, cardiovascular device, stent, defibrillator, graft, needle, catheter, scaffold, breast implant, or tissue engineering matrix. In another embodiment, the surface treatment is plasma treatment, ion or electron treatment, to induce electrostatic charges that inhibit biofilm formation and bacterial growth.

In another aspect, there is provided a device comprising nanoparticles, wherein said nanoparticles are positioned in one or more layers. In one embodiment, one or more layers are biodegradable and release nanoparticles upon degradation. In another embodiment, said layers comprise hydroxyapatite, wherein said layers degrade over time and release nanoparticles and/or microparticles of hydroxyapatite for stimulating bone formation adjacent to a surface of said device. In another embodiment, the layers comprise either externally or internally at least one antibiotic, growth factor, drug, or biofilm inhibitory agent, wherein said layers degrade and release said antibiotic, growth factor, drug, and/or biofilm inhibitory agent.

In another aspect, there is an implant comprising titanium, wherein zirconia coats at least one surface of said implant. In one embodiment, the implant is a dental implant or an abutment for a dental implant.

In another aspect, there is provide a method for coating a portion or surface with zirconia, comprising: depositing zirconia on said surface by one or more of ion beam deposition, electron beam deposition, pulsed laser deposition, thermal sputtering and deposition, RF sputtering, laser etching, glancing angle deposition, physical vapor deposition, molecular epitaxy and chemical vapor deposition, wherein said deposition produces a crystalline film. In one embodiment, the coating may be produced by self assembly and self formation during deposition. In another embodiment, the surface is a dental implant or an abutment for a dental implant. In another embodiment, the method further comprises depositing zirconia and at least one other coating agent and/or nanoparticle on at least one portion of said implant or abutment. In one embodiment, the surface is heated during said depositing or after said depositing in order to alter the crystallinity of said film.

In another aspect, there is provided a method of sterilizing a nanocomposite-coated device, comprising exposing said device to either ethylene oxide or gamma radiation.

In another aspect, there is provided a package comprising a nanocomposite-coated medical device, wherein said device is sealed in an airtight or vacuum packed container. In one embodiment, the medical device is a dental implant, an abutment for a dental implant, or any medical device.

In another aspect, there is provided a nanoparticle composition comprising: (a) a core made of one nanoparticle material; and (b) at least one layer surrounding said core, wherein said layer comprises a nanoparticle material that is not the same as said core. In one embodiment, the nanoparticle composition is deposited to form one or more layers on a medical device or implantable medical device for use in humans and/or animals and/or in vitro. In another embodiment, each layer may be comprised of different or similar heterogeneous nanoparticles. In other embodiments, each layer may be formed from any combination of natural or synthetic polymer, metal, metal oxide, oxide, metal nitride, borate, ceramic, zirconia, allograft hard tissue, allograft soft tissue, xenograft hard tissue, xenograft soft tissue, carbon nanostructure, carbon, glasses, or natural or biocompatible material. In another embodiment, the composition further comprising at least one bioactive agent. In another embodiment, the nanoparticle material and bioactive agent can be positioned in any orientation and place within said composition or on a surface layer of said composition.

In another aspect, there is provided a method for enhancing bone cell growth, comprising (a) depositing hydroxyapatite nanoparticles on a surface to create a surface coating; (b) exposing said surface coating to plasma treatment; and (c) culturing osteoblasts on said surface.

In another aspect, provided herein is a method for producing an orthodontic wire, comprising depositing zirconia on said wire by one or more of ion beam deposition, electron beam deposition, pulsed laser deposition, thermal sputtering and deposition, RF sputtering, laser etching, glancing angle deposition, physical vapor deposition, molecular epitaxy and chemical vapor deposition, wherein said deposition produces a crystalline film.

In another aspect, here is a method for producing an orthodontic wire, comprising depositing zirconia on said wire by pulsed laser deposition.

### BRIEF DESCRIPTION OF THE DRAWINGS

FIG. 1 illustrates polyurethane-hydroxyapatite nanocomposites created by in-situ polymerization. Observe polymeric pores and nanostructural hydroxyapatite at the surface.

FIG. 2 illustrates the number of osteoblast cells counted on the polymeric surfaces as a function of the titanium (Ti) control. The number of osteoblast cells on the polymeric films that were doped with hydroxyapatite (HA) nanoparticles and/or exposed to plasma treatment increased significantly as compared to the regular polymeric films.

FIG. 3 illustrates the enhanced growth of osteoblast cells on the polymeric surfaces exposed to plasma treatment and doped with HA nanoparticles (b) as compared to the regular Ti surfaces (a). The corrugated polymeric surfaces (doped with HA nanoparticles and plasma treated) (c) due to various modifications, have also been found to significantly enhance the proliferation of osteoblast cells.

FIG. 4 displays an application of a surface coating during manufacturing.

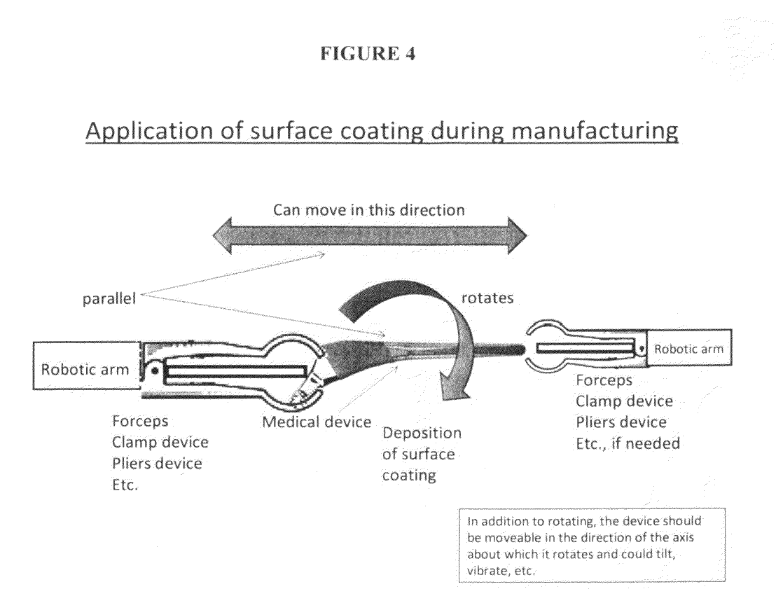

FIG. 5 displays an application of a surface coating during manufacturing.

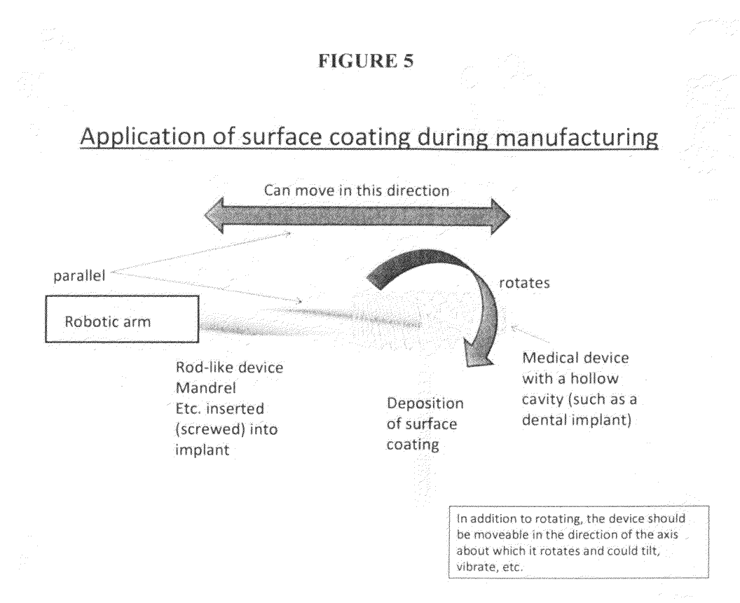

FIG. 6 displays an Atomic Force Microscopy (AFM) image of a polyurethane-hydroxyapatite nanocomposite film made by pulsed laser deposition.

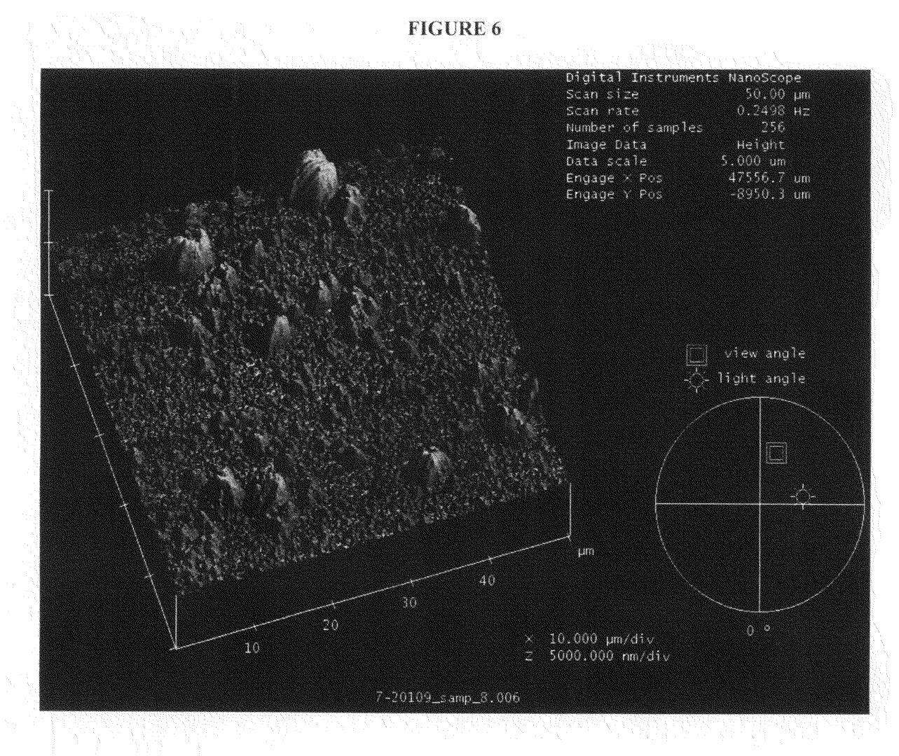

### DETAILED DESCRIPTION

Methodologies, materials, and devices provided herein relate to a coating that can be applied to the surface of an implant. More specifically, and as described below, a surface coating can be applied to any implant, such as a medical or dental implant, wherein the coating is bio-compatible, optionally bio-degradable, and facilitates surface adherence and proliferation of cells adjacent to and/or on an implant surface. The surface coating can also deliver drugs and/or bioactive agents that can lead to increased cell proliferation and bone mineralization at the implant surface. An illustrative surface coating can be applied to any tissue matrix or implant used for any internal/medical purpose. Surface coatings can also reduce and prevent growth of biofilm.

All technical terms used herein are terms commonly used in cell biology, biochemistry, molecular biology, and nanotechnology and can be understood by one of ordinary skill in the art to which this invention belongs. These technical terms can be found in the current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found in Current Protocols in Protein Science (J. E. Colligan et al. eds., Wiley & Sons); Current Protocols in Cell Biology (J. S. Bonifacino et al., Wiley & Sons) and Current Protocols in Immunology (J. E. Colligan et al. eds., Wiley & Sons.). Reagents, cloning vectors, and kits are available from commercial vendors such as BioRad, Stratagene, Invitrogen, ClonTech, and Sigma-Aldrich Co.

Cell culture methods are described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Other texts include Creating a High Performance Culture (Aroselli, Hu. Res. Dev. Pr. 1996) and Limits to Growth (D. H. Meadows et al., Universe Publ. 1974). Tissue culture supplies and reagents are available from commercial vendors such as Gibco/BRL, Nalgene-Nunc International, Sigma Chemical Co., and ICN Biomedicals.

Although this specification provides guidance to one of ordinary skill in the art, reference to technical literature, mere reference does not constitute an admission that the technical literature is prior art.

### A. Depositing of Surface Coating on a Material

A surface coating can be deposited on a material by any method known in the art. Non-limiting deposition methods include any one or more of ion beam deposition, electron beam deposition, pulsed laser deposition, thermal sputtering and deposition, RF sputtering, laser etching, glancing angle deposition, electrospray, chemical vapor deposition, physical vapor deposition, and molecular epitaxy. Of course, any technique by which molecules are delivered to a substrate of interest may be used. Coating can be done at the micro level or nano scale, depending on the intended use. Such methodologies are known in the art and may be found in, for example, Marc J. Madou's Fundamentals of Microfabrication, The Science of Miniaturization, 2.sup.nd Ed., including metal deposition at pages 344-357, or “Nanofabrication: Fundamentals and Applications” Ed.: Ampere A. Tseng, World Scientific Publishing Company (Mar. 4, 2008), ISBN 9812700765.

### B. Delivery of Surface-Coated Implant to a Host

A surface-coated implant can be delivered to a host organism by any suitable method known in the art. For example, and in no way limiting, a surface-coated implant can be delivered by epidermal translation, direct surgical placement, topical application, or oral administration. Delivery can be directed to any cell type or tissue in any organism.

In one embodiment, a surface-coated implant may be delivered to any eukaryotic cell or tissue of interest. In certain embodiments, a cell is a mammalian cell. Cells may be of human or non-human origin. For example, they may be of mouse, rat, or non-human primate origin. Exemplary cell types include but are not limited to endothelial cells, epithelial cells, neurons, hepatocytes, myocytes, chondrocytes, osteoblasts, osteoclasts, lymphocytes, macrophages, neutrophils, fibroblasts, keratinocytes, etc. Cells can be primary cells, immortalized cells, transformed cells, terminally differentiated cells, stem cells (e.g. adult or embryonic stem cells, hematopoietic stem cells), somatic cells, germ cells, etc. Cells can be wild type or mutant cells, e.g., they may have a mutation in one or more genes. Cells may be quiescent or actively proliferating. Cells may be in any stage of the cell cycle. In some embodiments, cells may be in the context of a tissue. In some embodiments, cells may be in the context of an organism.

Cells can be normal cells or diseased cells. In certain embodiments, cells are cancer cells, e.g. they originate from a tumor or have been transformed in cell culture (e.g. by transfection with an oncogene). In certain embodiments, cells are infected with a virus or other infectious agent(s). A virus may be, e.g. a DNA virus, RNA virus, retrovirus, etc. For example, cells can be infected with a human pathogen such as a hepatitis virus, a respiratory virus, human immunodeficiency virus, etc.

Cells can be cells of a cell line. Exemplary cell lines include HeLa, CHO, COS, BHK, NIH-3T3, HUVEC, etc. For an extensive list of cell lines, one of ordinary skill in the art may refer to the American Type Culture Collection catalog (ATCC®, Manassas, Va.).

In some embodiments, speed or delivery rate of nanoparticles, drugs, and/or bioactive agents to a cell type and/or tissue may be increased by exposing said cell and/or tissue comprising a surface-coated implant to radiation, which permits faster penetration of the host cell and/or tissue. Any suitable radiation technique may be used, including laser radiation and electromagnetic radiation.

### C. Illustrative Products

The nanoparticle and/or microparticle compositions provided herein may be used in a variety of products, including but not limited to implants, devices, compositions, nutraceuticals, topicals, gels, creams, kits, reagents, implants, scaffolds, cell culture dishes, and related tools.

For example, nanoparticle and/or microparticle compositions may be used to coat a variety of implants, including but not limited to an orthopedic implant, dental implant, veterinary prosthetic device, graft, needle, bone material, contact lens, catheter, ear tube, endotracheal tube, stent, shunt, scaffold, tissue engineering matrix, breast implant, allograft hard tissue, allograft soft tissue, xenograft hard tissue, xenograft soft tissue, polymeric mesh, hip implant, knee implant, shoulder implant, plate, pin, screw, wire, rod, or ceramic mesh.

For instance, in the case of a dental implant, any component of a dental implant may be coated or otherwise comprise nanoparticle and/or microparticle compositions. Dental implant components include but are not limited to an abutment, healing screw, and cover screw. Other dental applications include archwires used in orthodontics and removable partial denture clasps and connectors used in dentistry which may be coated with a nanoparticle or microparticle composition.

Specific examples are presented below of methods. They are exemplary and not limiting.

### EXAMPLE 1

### Preparation of Polyurethane-Hydroxyapatite Composite Materials

Polyurethane-hydroxyapatite (HA) composite materials are prepared by in-situ polymerization. The nanomaterials are mixed in the solvent of the polymeric scaffold and deposited by air spraying.

Similarly, for industrial applications, the composite films can be deposited by e-beam deposition, ion beam deposition, pulsed laser deposition, electrospray, or any other method known in the art, on the implant surfaces.

The coating can be exposed to any type of plasma discharge and covered with growth factors, proteins, amino acids, drugs, hydroxyapatite, or any other bioactive agent.

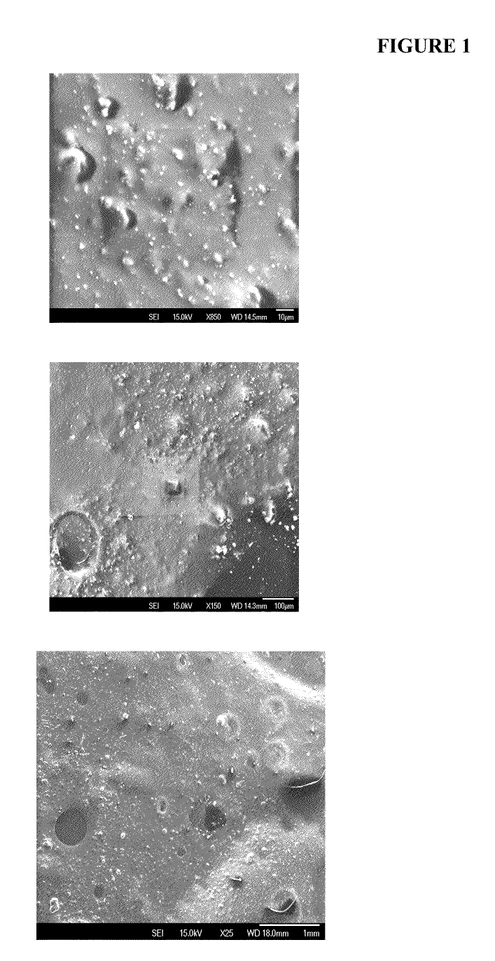

FIG. 1 presents a surface created by the deposition of the polymeric/nano-hydroxyapatite nanocomposites.

### EXAMPLE 2

### Cell Growth Assay on Coated and Non-Coated Surfaces

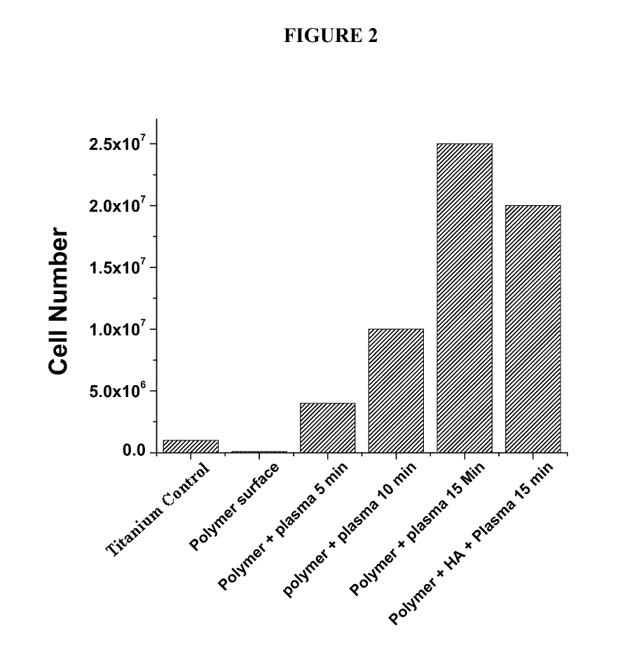

Osteoblast cells were incubated in tissue culture on top of the coatings and controls for 7 days at 37 deg. C. At day 0, at the start of the experiment, the number of cells was 10⁵. The control surfaces were a roughened titanium surface and an untreated polymer surface. After 7 days in tissue culture, osteoblasts were counted using standardized cell counting methods. The number of cells on the untreated polymer surface was 10⁵. The number of cells on the second control (titanium surface) was 10⁶. The results for the polyurethane coatings and HA are shown in FIG. 2.

As show in FIG. 2, there is a significant increase in the number of cells on the plasma treated polymer coatings, as well as the polymer treated and HA coatings. The polymer surfaces which were plasma treated between 10 and 15 minutes demonstrated approximately 20 times more cells per unit area versus the controls. The plasma treated polymer surface with hydroxyapatite nano-particles exhibited 20 times the number of cells per unit area versus the controls. These results indicate a significant increase in cellular adherence and growth of cells (osteoblasts) to plasma treated polymer surface coatings (with or without hydroxyapatite) versus roughened conventional titanium surface coatings used for surgical implantation in humans.

### EXAMPLE 3

### Visualization of Bone Cells on Various Surfaces

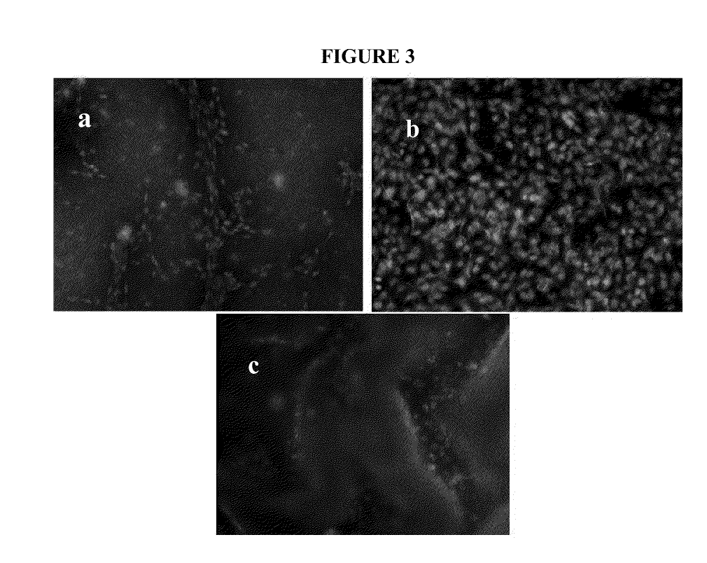

FIG. 3 shows enhanced growth of bone cells on the polymeric surfaces exposed to plasma and doped with HA nanoparticles (b) as compared to the regular Ti surfaces (a). The corrugated polymeric surfaces (doped with HA nanoparticles and plasma treated) (c) due to various modifications, have also been found to enhance significantly the proliferation of osteoblast cells. The visualization was done with optical microscopy and cellular staining.

### EXAMPLE 4

### Zirconia Surface Deposition

Zirconia deposition can be achieved by pulsed laser deposition, e-beam deposition, or any of the processes involving atomic or molecular deposition. Many surfaces can be used for zirconia deposition, including but not limited to an orthopedic implant, dental implant, abutement for a dental implant, orthodontic archwires, removable partial denture clasps, and connectors used in dentistry.

What is claimed is:

1. A coating comprising micro and/or nano materials deposited on a surface.

2. The coating of claim 1, wherein said coating is deposited onto a device.

3. The coating of claim 2, wherein said device is a medical device.

4. The coating of claim 3, wherein said medical device is implantable.

5. The coating of claim 2, wherein said device is delivered into a host organism or used in vitro.

6. The coating of claim 5, wherein said host organism is a human or animal.

7. The coating of claim 2, wherein said coating covers at least a portion of said device.

8. The coating of claim 1, wherein said coating comprises natural or synthetic polymer, metal, metal oxide, oxide, metal nitride, borate, ceramic, zirconia, allograft hard tissue, allograft soft tissue, xenograft hard tissue, xenograft soft tissue, carbon nanostructure, carbon, glasses, natural or biocompatible material.

9. The coating of claim 1, wherein said micro and/or nano materials comprise material having a size from about 0.5 nm to about 50 mm.

10. The coating of claim 1, wherein said coating is capable of performing at least one of preventing oxidation; decreasing toxicity; treating infection, preventing infection; promoting cell adhesion; preventing biofilm formation, inhibiting biofilm formation; promoting cell proliferation; promoting binding with a biological or non-biological system, increasing or decreasing a cell function; delivering a drug and/or bioactive agent, or ensuring a better integration of a material into the host tissue

11. The coating of claim 1, wherein said coating is deposited by one or more of ion beam deposition, electron beam deposition, pulsed laser deposition, thermal sputtering and deposition, RF sputtering, laser etching, glancing angle deposition, electrospray, chemical vapor deposition, physical vapor deposition, molecular epitaxy.

12. The coating of claim 11, wherein said coating may be produced by self assembly and self formation during deposition.

13. The coating of claim 1, wherein at least one surface of said coating may undergo plasma/ion treatment to induce the formation of surface charges that enhance the binding of bioactive agents, growth factors, and/or drugs, promote cell adhesion and proliferation, and/or increase the hydrophilic nature of the surface.

14. The coating of claim 1, wherein said coating comprises nanoparticles and microparticles.

15. The coating of claim 1 , wherein said coating comprises one or more layers of nanoparticles and/or microparticles.

16. The coating of claim 15, wherein said one or more layers comprises a single type of nanoparticle and/or microparticle, or a combination of more than one type of nanoparticle and/or microparticle.

17. The coating of claim 16, wherein said one or more layers comprises silver nanoparticles.

18. The coating of claim 16, wherein said one or more layers comprises a combination of metal nanoparticles, metal oxides, carbon nanotubes, polymeric nanoparticles, ceramics, calcium phosphate, collagen, and/or hydroxyapatite nanoparticles.

19. The coating of claim 15, wherein said coating is biodegradable and/or biocompatible.

20. The coating of claim 19, wherein said nanoparticles are released from said nanoparticle composition as each layer degrades.

21. The coating of claim 15, wherein a drug, growth factor, and/or bioactive agent is deposited within at least one layer and/or on the surface layer of said coating.

22. The coating of claim 14, wherein said nanoparticles comprise gold, silver, metals, oxides, carbon nanostructures (single, double, multi walled nanotubes, graphenes, fullerenes, nanofibers), hydroxyapatite, zirconia, natural or synthetic polymers, ceramics, or metal oxide.

23. The coating of claim 3, wherein said medical device is an orthopedic implant, dental implant, veterinary prosthetic device, graft, needle, bone material, contact lens, catheter, defibrillator, pacemaker, ear tube, endotracheal tube, stent, shunt, scaffold, or tissue engineering matrix, breast implant, allograft hard tissue, allograft soft tissue, xenograft hard tissue, xenograft soft tissue, polymeric mesh, or ceramic mesh.

24. The coating of claim 23, wherein said orthopedic implant is a hip implant, knee implant, shoulder implant, plate, pin, screw, wire, or rod.

25. The coating of claim 23, wherein said dental implant is an abutment, healing screw, or cover screw.

26. The coating of claim 23, wherein said veterinary prosthetic device is an implant, pin, screw, plate, or rod.

27. The coating of claim 3, wherein said medical device comprises plasmids, genes, nucleic acids, or a DNA or RNA virus.

28. The coating of claim 16, wherein said one or more layers comprise at least one of a protein, amino acid, collagen, enzyme, nucleic acid, bioactive agent, growth factor, drug, antibiotic, nucleic acid, hormone, antibody, or agent that inhibits biofilm formation and may be released as layer(s) degrade.

29. The coating of claim 28, wherein said growth factor is a bone morphogenic protein capable of promoting bone formation adjacent to or on the surface of a device.

30. The coating of claim 28, wherein said bioactive agent is in or on the surface coating of a medical device and affects adjacent tissue or cells in at least one or more of bone formation, protein synthesis, gene expression, cell proliferation, mitosis, DNA transcription, hormone production, enzyme production, cell death, gene delivery, or drug delivery.

31. The coating of claim 28, wherein said bioactive agent may be linked to said nanoparticles.

32. The method of claim 31, wherein said linkage may be a covalent, ionic, hydrogen bond, sulfide bond, or polar covalent bond.

33. The coating of claim 1, wherein a structured surface can be prepared by at least one of flame spraying, acid etching, grit blasting, casting-in, forging-in, laser texturing, micromachining, plasma treatment, ion bombardment, physical vapor deposition, or chemical vapor deposition

34. A method for inhibiting biofilm formation on a medical implant, comprising coating said implant with an agent(s) that prevents biofilm formation and/or growth of bacteria.

35. The method of claim 34, wherein said biofilm is a bacterial, fungal, or protozoan biofilm.

36. The method of claim 34, wherein said medical implant is an orthopedic or dental implant, graft, needle, bone material, contact lens, catheter, ear tube, endotracheal tube, stent, shunt, breast implant, scaffold, allograft hard tissue, allograft soft tissue, xenograft hard tissue, xenograft soft tissue or tissue engineering matrix.

37. The method of claim 34, wherein said agent is triclosan, iodine, silver, phenol, chloride compounds, fluoride compounds, iodine, quaternary ammonium compounds, chlorhexidine, antibiotic, antifungal agent, or any other agent that inhibits biofilm formation and/or growth.

38. A method for inhibiting microbial colonization on a medical device or implant, comprising coating said device or implant with an agent or surface treatment that prevents microbial colonization.

39. The method of claim 38, wherein said device or implant is a dental implant, healing screw or cover screw for a dental implant, orthopedic implant, veterinary implant, cardiovascular device, stent, defibrillator, graft, needle, catheter, scaffold, breast implant, or tissue engineering matrix.

40. The method of claim 38, wherein said surface treatment is plasma treatment, ion or electron treatment, to induce electrostatic charges that inhibit biofilm formation and bacterial growth.

41. A device comprising nanoparticles, wherein said nanoparticles are positioned in one or more layers.

42. The device of claim 41, wherein said one or more layers are biodegradable and release nanoparticles upon degradation.

43. The device of claim 42, wherein said layers comprise hydroxyapatite, wherein said layers degrade over time and release nanoparticles and/or microparticles of hydroxyapatite for stimulating bone formation adjacent to a surface of said device.

44. The device of claim 42, wherein said layers comprise either externally or internally at least one antibiotic, growth factor, drug, or biofilm inhibitory agent, wherein said layers degrade and release said antibiotic, growth factor, drug, and/or biofilm inhibitory agent.

45. An implant comprising titanium, wherein zirconia coats at least one surface of said implant.

46. The implant of claim 45, wherein said implant is a dental implant or an abutment for a dental implant.

47. A method for coating a portion or surface with zirconia, comprising:
depositing zirconia on said surface by one or more of ion beam deposition, electron beam deposition, pulsed laser deposition, thermal sputtering and deposition, RF sputtering, laser etching, glancing angle deposition, physical vapor deposition, molecular epitaxy and chemical vapor deposition, wherein said deposition produces a crystalline film.

48. The method of claim 47, wherein said coating may be produced by self assembly and self formation during deposition.

49. The method of claim 47, wherein said surface is a dental implant, an abutment for a dental implant, an orthodontic archwire, a connector used in dentistry, or a removable partial denture clasp.

50. The method of claim 47, further comprising depositing zirconia and at least one other coating agent and/or nanoparticle on at least one portion of said implant or abutment.

51. The method of claim 47, wherein said surface is heated during said depositing or after said depositing in order to alter the crystallinity of said film.

52. A method of sterilizing a nanocomposite-coated device, comprising exposing said device to either ethylene oxide or gamma radiation.

53. A package comprising a nanocomposite-coated medical device, wherein said device is sealed in an airtight or vacuum packed container.

54. The package of claim 53, wherein said medical device is a dental implant, an abutment for a dental implant, or any medical device of claim 23.

55. A nanoparticle composition comprising:
(a) a core made of one nanoparticle material; and
(b) at least one layer surrounding said core, wherein said layer comprises a nanoparticle material that is not the same as said core.

56. The nanoparticle composition of claim 55, wherein said nanoparticle composition is deposited to form one or more layers on a medical device or implantable medical device for use in humans and/or animals and/or in vitro.

57. The nanoparticle composition of claim 55, wherein each layer may be comprised of different or similar heterogeneous nanoparticles.

58. The nanoparticle composition of claim 55, wherein each layer may be formed from any combination of natural or synthetic polymer, metal, metal oxide, oxide, metal nitride, borate, ceramic, zirconia, allograft hard tissue, allograft soft tissue, xenograft hard tissue, xenograft soft tissue, carbon nanostructure, carbon, glasses, or natural or biocompatible material.

59. The nanoparticle composition of claim 55, further comprising at least one bioactive agent.

60. The nanoparticle composition of claim 59, wherein said nanoparticle material and said bioactive agent can be positioned in any orientation and place within said composition or on a surface layer of said composition.

61. A method for enhancing bone cell growth, comprising
(a) depositing hydroxyapatite nanoparticles and polymer nanoparticles on a surface to create a surface coating;
(b) exposing said surface coating to plasma treatment; and
(c) culturing osteoblasts on said surface.

62. A method for surface coating an orthodontic wire, removable partial denture clasp, or connector used in dentistry, comprising depositing zirconia on said wire, removable partial denture clasp, or connector by one or more of ion beam deposition, electron beam deposition, pulsed laser deposition, thermal sputtering and deposition, RF sputtering, laser etching, glancing angle deposition, physical vapor deposition, molecular epitaxy and chemical vapor deposition, wherein said deposition produces a crystalline film.

63. A method for producing an orthodontic wire, removable partial denture clasp, or connector used in dentistry, comprising depositing zirconia by pulsed laser deposition on said wire, removable partial denture clasp, or connector.
